(19) United States
(12) Patent Application Publication
(10) Pub. No.: US 2013/0053827 A1
(43) Pub. Date: Feb. 28, 2013

(54) Guide Wire Insertion Aid

(71) Applicants: Mark Schippel, Rangsdorf (DE); Roland Eichberg, Berlin (DE); Siegfried Voigt, Berlin (DE); Klaus Bartels, Berlin (DE)

(72) Inventors: Mark Schippel, Rangsdorf (DE); Roland Eichberg, Berlin (DE); Siegfried Voigt, Berlin (DE); Klaus Bartels, Berlin (DE)

(73) Assignee: BIOTRONIK SE & CO. KG, Berlin (DE)

(21) Appl. No.: 13/590,563

(22) Filed: Aug. 21, 2012

Related U.S. Application Data
(60) Provisional application No. 61/529,261, filed on Aug. 31, 2011.

(51) Int. Cl. A61M 25/09

(52) U.S. Cl. 604/528

(57) ABSTRACT

A guide wire insertion aid for attaching onto a proximal end of a catheter or an implantable electrode line and for facilitating the insertion of a guide wire, formed as a horn- or funnel-shaped body which has a tubular fixing section and an expanding section extending therefrom, wherein the fixing section has an inner wall without contour and is slotted and elastically expandable at least over the larger portion of its longitudinal extension.

### CROSS-REFERENCE TO RELATED APPLICATIONS

This patent application claims the benefit of co-pending U.S. Provisional Patent Application No. 61/529,261, filed on Aug. 31, 2011, which is hereby incorporated by reference in its entirety.

### TECHNICAL FIELD

The present invention relates to a guide wire insertion aid for attaching onto a proximal end of a catheter or an implantable electrode line and for facilitating the insertion of a guide wire.

### BACKGROUND

Handling catheters or catheter-like instruments and, in particular, implantable electrode lines as they are used in connection with cardiac pacemakers or implantable cardioverters is very common in clinical practice. In more demanding applications, the use typically takes place in connection with guide wires which allow precise guiding and positioning of the respective catheter or electrode line even if branched vessels or cavities with complicated shapes have to be passed and an exactly predetermined position is to be reached.

Since inserting the guide wire into the lumen provided for receiving the guide wire in the respective catheter or the electrode line is already a delicate matter, special insertion aids were developed to make it easier for the surgeon to find the proximal end of the catheter or the electrode line with the guide wire. However, such insertion aids are adapted in terms of shape to the special type of connector of the electrode line and, therefore, are suited in each case only for a particular electrode line. Moreover, from U.S. Pat. No. 6,652,492, a continuously slotted insertion aid became known. The disadvantage of this insertion aid is that during the handling by the surgeon—which often requires insertion of the guide wire multiple times into the electrode line—said insertion aid can get lost and then has to be replaced with a new sterile insertion aid. This is disturbing for the surgical process and, of course, also requires additional logistic effort.

The present invention is directed toward overcoming one or more of the above-identified problems.

### SUMMARY

It is therefore an object of the present invention to provide an improved insertion aid of the aforementioned type which is in particular characterized by an easy and safe handling and usability for different types of catheters and electrode lines.

An object is achieved by the insertion aid with the features of the independent claim(s). Advantageous further embodiments of the present invention are the subject matter of the dependent claims.

The present invention incorporates the idea to design the insertion aid as a component which adapts automatically to a certain extent to different proximal ends of catheters or electrode lines. It further includes the idea to configure the fixing section of the insertion aid for this purpose in a radially elastically expandable manner. Additionally, the present invention involves the idea to provide a slot in the fixing section, at least over the larger portion of the fixing section's longitudinal extension, which slot partially provides for the desired expandability in connection with a sufficient holding force on the catheter end or line end. Finally, the present invention involves the idea to keep the inner wall of the fixing section free of (annular) edges, steps, etc. which, in turn, is only made possible or at least facilitated by the elastic expandability and by providing said slot.

The proposed inventive solution is in particular advantageous with respect to the following application aspects:
During the implantation of an electrode, the insertion aid is attached several times onto the connector pin so as to carry out, for example, a change of the guide wire or mandrin (e.g., harder mandrins, pre-bent mandrins, etc.).
To frequently remove the insertion aid from the connector pin, for example, to be able to calibrate an electrode in the patient. It is of advantage here that, due to the non-continuous slot, the insertion device does not have to be removed from the implantation system, i.e., does not have to be detached from the mandrin, or cannot fall into the surgical wound. The inventive insertion aid stays where it is needed.
When fixing “screw electrodes”, the inventive insertion aid is simply drawn off of the collector pin and remains hanging on the protruding mandrin. Thus, a screwing tool can be attached to the connector pin and it is ensured that the mandrin can push the electrode against the heart wall during fixation.
Owing to the elastic expandability, the inventive insertion aid can be retained in sufficiently secure manner on the different (plug) connectors, despite the dimensional differences of the connectors which are suitable for said insertion aid.
The substantially “smooth” design of the inner wall of the section, without inner edges or steps, ensures that color markings attached on the plug connectors remain undamaged during the handling required for the implantation.

The above-mentioned technical advantages lead, among other things, to the following economic advantages:
For electrode lines or catheters with different connections, an insertion aid can be developed, produced and delivered as a single embodiment, whereby manufacturing costs, sales costs and logistic costs are reduced on the side of the manufacturer.
On the side of the user, this results in a corresponding simplification with respect to storage and pre-operative planning, and the user too benefits from the low development and manufacturing costs.

In one embodiment of the present invention, at least the slotted portion of the fixing section consists of a dimensionally elastic plastic. In a further configuration, the insertion aid, as a whole, is made as one piece from plastic which is dimensionally elastic to a certain degree.

As an alternative, an embodiment is possible, wherein a radially acting spring element is provided in the slotted portion of the fixing section. Such an embodiment is slightly costlier to produce, but can be designed with respect to its mechanical properties largely independent of the use of particular plastic materials.

In a further embodiment of the proposed guide wire insertion aid, the fixing section has a longitudinally extending slot. On the other hand, it can be provided that the fixing section has a spirally extending slot. Finally, in further embodiments, it is also possible that the fixing section has a plurality of slots which optionally can have different dimensions and/or shapes. One skilled in the art will appreciate that many slot configurations can be implemented without departing from the spirit and scope of the present invention.

In a further embodiment of the present invention, the inner wall of the fixing section is proximally bounded by a limit stop for the inserted end of the catheter or the electrode line.

Preferably, at its end adjacent to the fixing section, the inner diameter of the expanding section is smaller than the inner diameter of the fixing section and, in fact, is preferably adjusted to the diameter of a lumen provided in the catheter or in the electrode line for receiving the guide wire. Thus, the limit stop is formed by the end of the expansion section.

Another embodiment of the present invention provides that the inner diameter of the fixing section is adjusted (with a slight undersize) to the connector of a standard electrode line, in particular, for example, a DF-4 and/or IS-1 and/or IS-4 and/or DF-1 connector. Apart from the mentioned standard connection, the insertion aid can be configured, if required, to fit for further connections.

Further features, aspects, objects, advantages, and possible applications of the present invention will become apparent from a study of the exemplary embodiments and examples described below, in combination with the Figures, and the appended claims.

### DESCRIPTION OF THE DRAWINGS

Apart from that, advantages and usefulness of the invention arise from the following description of an exemplary embodiment based on the Figures. In the drawings:

FIG. 1 shows a perspective view of a guide wire insertion aid;

FIGS. 2A and 2B show a top view (from the free end of the fixing section) and a longitudinal sectional view, respectively, of the guide wire insertion aid according to FIG. 1; and

FIG. 3 shows an illustration for clarification of the use of the guide wire insertion aid according to FIGS. 1 and 2A-2B.

### DETAILED DESCRIPTION

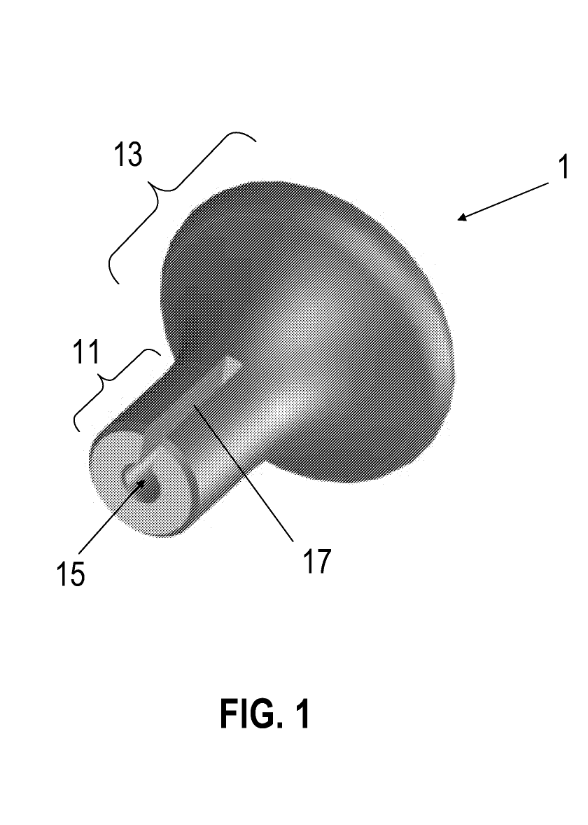

FIG. 1 shows s perspective view of a one-piece guide wire insertion aid 1 made from a dimensionally elastic plastic, the general basic shape of which is the shape of a horn, and which has a fixing section 11 and an expanding section 13 adjacent thereto, as well as a central longitudinal through-opening 15. Also shown is a slot 17 which extends longitudinally over almost the entire length of the fixing section 11.

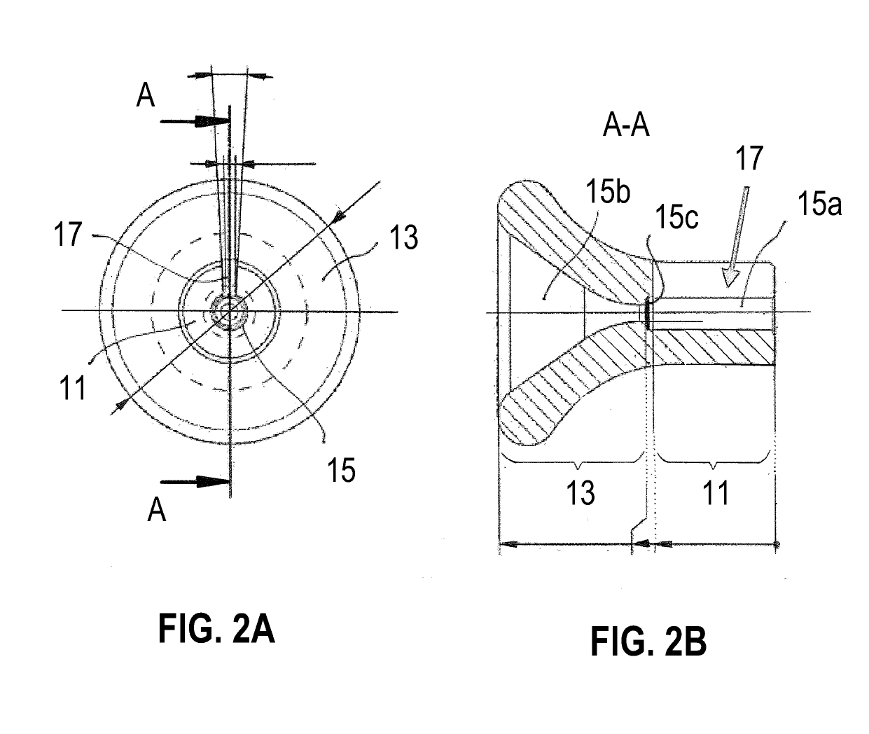

FIGS. 2A and 2B show this structure in more detail in a top view and a longitudinal sectional view, respectively. It is in particular also shown here that the centric through-opening 15 comprises, in the fixing section 11, a first cylindrical partial section 15a which has a wall without contours and, in the expanding section 13, a second, horn-like expanding section 15b. At the border to the adjacent end of the fixing section 15a, the diameter of the expanding section 15b is smaller than the one of the first section 15a so that an annular stop 15c is formed here. This configuration of the central through-opening 15 serves, on the one hand, for limiting the insertion depth of an electrode line connector or catheter end to be inserted in the fixing section 11 and, on the other hand, to securely direct a guide wire inserted from the wide end of the expanding section 13 into a lumen provided for the guide wire in the electrode line or the catheter.

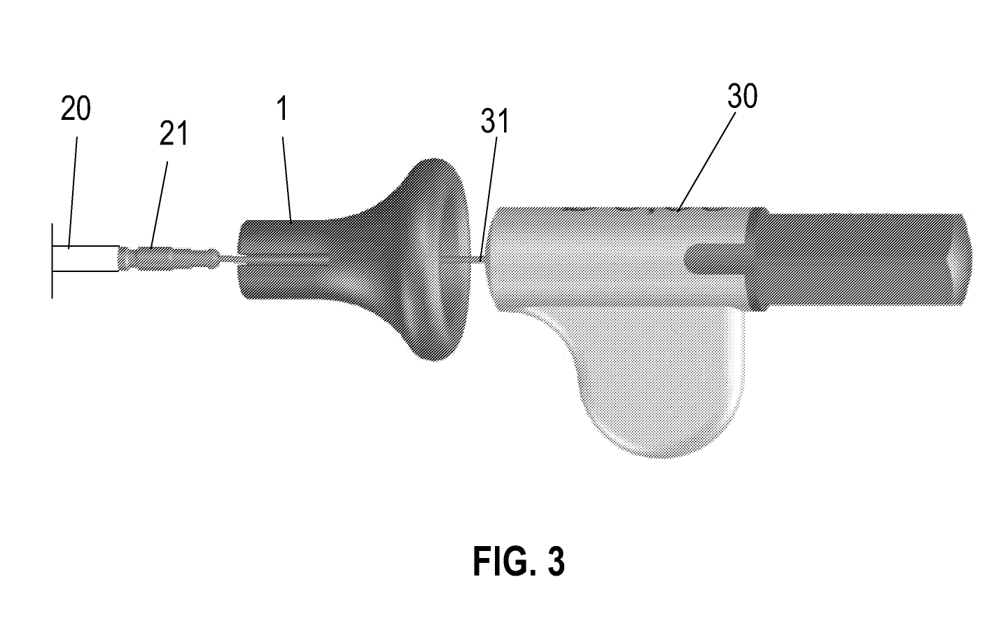

FIG. 3 shows the guide wire insertion aid 1 in use at a plug connector 21 of an electrode line 20 and with the inserted guide wire 31 of a guide wire arrangement 30.

The embodiment described herein is not limited to this example and the aspects emphasized above, but is also possible in a plurality of modifications which lie within the scope of persons skilled in the art. Thus, in particular, other shapes of the expanding section 13 of the insertion aid 1 (including, in particular, a funnel shape with truncated cone-shaped wall), multi-part embodiments and many different variations with respect to longitudinal and lateral dimensions, wall thicknesses, etc. are to be considered as lying within the scope of the present invention.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/we claim:

1. A guide wire insertion aid for attaching onto a proximal end of a catheter or an implantable electrode line and for facilitating the insertion of a guide wire, the guide wire insertion comprising:
a horn- or funnel-shaped body which has a tubular fixing section and an expanding section extending therefrom, wherein the fixing section has an inner wall without contour and is slotted and elastically expandable at least over a larger portion of its longitudinal extension.

2. The guide wire insertion aid according to claim 1, wherein at least the slotted portion of the fixing section comprises a dimensionally elastic plastic.

3. The guide wire insertion aid according to claim 2, designed as one piece and made from a dimensionally elastic plastic.

4. The guide wire insertion aid according to claim 1, wherein in the slotted portion of the fixing section, a radially acting spring element is provided.

5. The guide wire insertion aid according to claim 1, wherein the fixing section has a longitudinally extending slot.

6. The guide wire insertion aid according to claim 1, wherein the fixing section has a spirally extending slot.

7. The guide wire insertion aid according to claim 1, wherein the inner wall of the fixing section is proximally bordered by a limit stop for the inserted end of the catheter or the electrode line.

8. The guide wire insertion aid according to claim 7, wherein the inner diameter of the expanding section is smaller at its end adjacent to the fixing section than the inner diameter of the fixing section, and the limit stop is thus formed by the end of the expanding section.

9. The guide wire insertion aid according to claim 1, wherein the inner diameter of the fixing section is adjusted with undersize to the connector of a standard electrode line including a DF-4 and/or IS-1 and/or IS-4 and/or DF-1 connector.
